(19) United States
(12) Patent Application Publication
(10) Pub. No.: US 2026/0076678 A1
(43) Pub. Date: Mar. 19, 2026

(54) MEDICAL DEVICES FOR USE IN DELIVERING OCCLUSIVE IMPLANTS

(71) Applicant: BOSTON SCIENTIFIC SCIMED, INC., Maple Grove, MN (US)

(72) Inventors: Amber Joy Younggren, Oak Grove, MN (US); Philip Andrew Litecky, Forest Lake, MN (US); Joshua Matthew Tracy, St Michael, MN (US); Caleb Traut, Minneapolis, MN (US); David John Onushko, Maple Grove, MN (US)

(73) Assignee: BOSTON SCIENTIFIC SCIMED, INC., Maple Grove, MN (US)

(21) Appl. No.: 19/323,191

(22) Filed: Sep. 9, 2025

Related U.S. Application Data
(60) Provisional application No. 63/694,398, filed on Sep. 13, 2024.

(51) Int. Cl. A61B 17/12; A61B 17/00

(52) U.S. Cl. CPC A61B 17/12022; A61B 2017/00526; A61B 2017/1205

(57) ABSTRACT

Medical devices as well as methods for manufacturing medical devices are disclosed. An example medical device may include a medical device for use in delivering an occlusive implant. The medical device may include a core shaft having a distal end region. A tip member may be coupled to the distal end region. The tip member may include a collar region and an occlusive implant securing region. The collar region may have a plurality of apertures formed therein. The collar region may be bonded to the distal end region such that a portion of the distal end region of the core shaft forms a mechanical bond with the collar region. A sleeve may be disposed over the collar region.

### CROSS REFERENCE TO RELATED APPLICATIONS

The present application claims the benefit of U.S. Provisional Patent Application Ser. No. 63/694,398, filed Sep. 13, 2024, the disclosure of which is incorporated herein by reference.

### TECHNICAL FIELD

The present disclosure pertains to medical devices, and methods for manufacturing medical devices. More particularly, the present disclosure pertains to medical devices for use in delivering occlusive implants.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A medical device for use in delivering an occlusive implant is disclosed. The medical device comprises: a core shaft having a distal end region; a tip member coupled to the distal end region, the tip member having a collar region and an occlusive implant securing region; wherein the collar region includes a plurality of apertures formed therein; wherein the collar region is bonded to the distal end region such that a portion of the distal end region of the core shaft forms a mechanical bond with the collar region; and a sleeve disposed over the collar region.

Alternatively or additionally to any of the embodiments above, the core shaft includes a coil.

Alternatively or additionally to any of the embodiments above, the medical device further comprises a braid disposed over the coil.

Alternatively or additionally to any of the embodiments above, the coil includes an open pitch region along at least a section of the distal end region.

Alternatively or additionally to any of the embodiments above, the collar region extends along an outer surface of the distal end region of the core shaft.

Alternatively or additionally to any of the embodiments above, the collar region is thermally bonded with the distal end region of the core shaft.

Alternatively or additionally to any of the embodiments above, the mechanical bond with the collar region is formed by the core shaft extending at least partially into at least some of the apertures of the collar region.

Alternatively or additionally to any of the embodiments above, the collar region extends along an inner surface of the distal end region of the core shaft.

Alternatively or additionally to any of the embodiments above, the collar region is threadably engaged with the inner surface of the distal end region of the core shaft.

Alternatively or additionally to any of the embodiments above, the apertures include one or more round or oval openings.

Alternatively or additionally to any of the embodiments above, the apertures include one or more slots oriented substantially normal relative to a longitudinal axis of the collar region.

Alternatively or additionally to any of the embodiments above, the apertures include one or more slots angled relative to a longitudinal axis of the collar region.

A medical device for use in delivering an occlusive implant is disclosed. The medical device comprises: a core shaft having a distal end region; wherein the core shaft includes a coil; wherein the coil includes an open pitch section adjacent to the distal end region of the core shaft; a tip member coupled to the distal end region, the tip member having a collar region configured to attach the tip member to the core shaft and an occlusive implant securing region configured to be attached to an occlusive implant; wherein the collar region is bonded to the distal end region such that a portion of the distal end region of the core shaft forms a mechanical bond with the collar region; and a sleeve disposed over the collar region.

Alternatively or additionally to any of the embodiments above, the medical device further comprises a braid disposed over the coil.

Alternatively or additionally to any of the embodiments above, the collar region extends along an outer surface of the distal end region of the core shaft.

Alternatively or additionally to any of the embodiments above, the collar region is thermally bonded with the distal end region of the core shaft.

Alternatively or additionally to any of the embodiments above, the collar region extends along an inner surface of the distal end region of the core shaft.

Alternatively or additionally to any of the embodiments above, the collar region is threadably engaged with the open pitch section of the coil.

Alternatively or additionally to any of the embodiments above, the collar region includes a plurality of apertures formed therein.

A method for manufacturing a medical device is disclosed. The method comprises: engaging a collar region of a tip member with a distal end region of a core shaft; wherein the collar region includes a plurality of apertures formed therein; and bonding the collar region with the distal end region such that a portion of the distal end region of the core shaft extends into at least some of the apertures and forms a mechanical bond with the collar region.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example delivery system.

FIG. 2 is a side view of an example delivery system.

FIG. 3 is a partial cross-sectional side view of a portion of an example medical device.

FIG. 4 is a perspective view of an example tip member.

FIG. 5 is a side view of a portion of an example medical device.

FIG. 6 is a perspective view of an example tip member.

FIG. 7 is a perspective view of an example tip member.

FIG. 8 is a side view of an example tip member.

FIG. 9 is a side view of a portion of an example medical device.

FIG. 10 is a partial cross-sectional side view of a portion of an example medical device.

FIG. 11 is a side view of an example tip member.

FIG. 12 is a side view of a portion of an example medical device.

FIG. 13 is a partial cross-sectional side view of a portion of an example medical device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

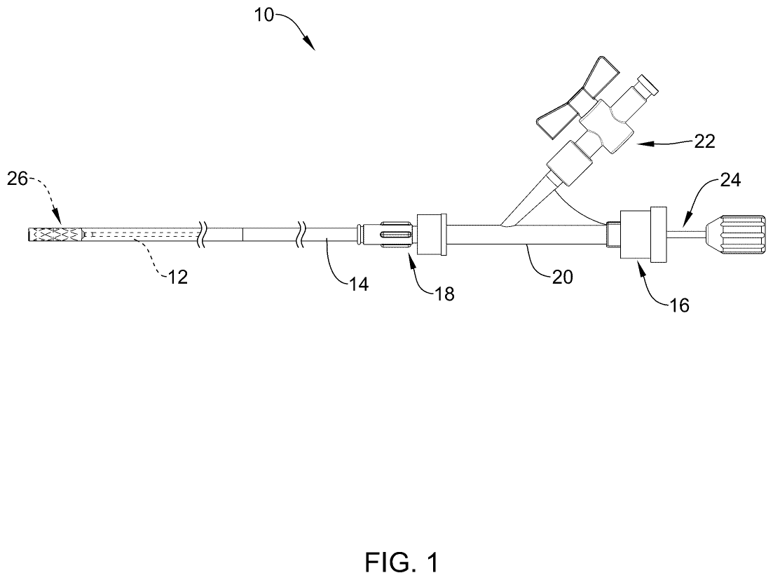

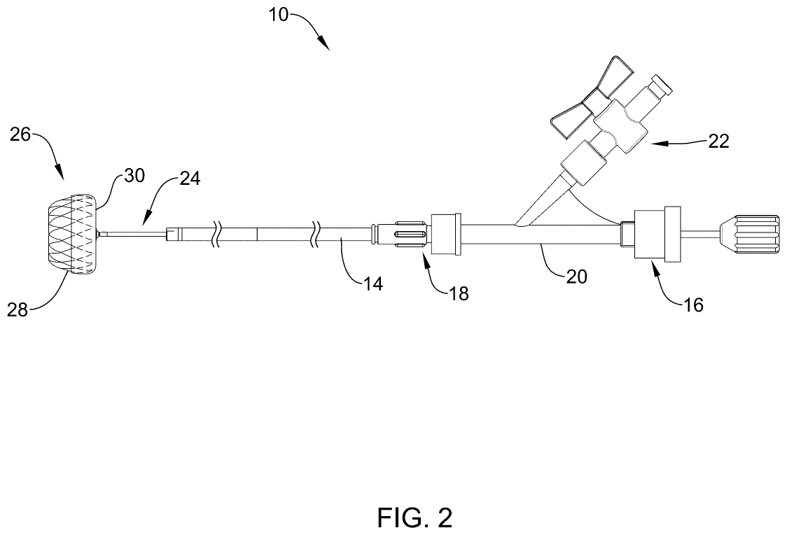

FIGS. 1-2 schematically illustrate selected components and/or arrangements of an occlusive implant system. It should be noted that in any given figure, some features of the occlusive implant system may not be shown, or may be shown schematically, for simplicity. Additional details regarding some of the components of the occlusive implant system may be illustrated in other figures in greater detail. The occlusive implant system may be used to deliver and/or deploy a variety of medical implants (e.g., a cardiovascular implant, an occlusive implant, etc.) to one or more locations within the anatomy, including but not limited to, in some embodiments, the heart and/or the left atrial appendage. In the interest of clarity, the following discussion refers to an occlusive implant, but other medical implants may be used and/or considered with the occlusive implant system.

The occlusive implant system may include a delivery system 10 including a delivery sheath 14 having a delivery lumen 12 extending proximally from a distal end of the delivery sheath 14. In one example, the delivery lumen 12 extends from a proximal opening to a distal opening of the delivery sheath 14. The delivery system 10 may include a proximal hub 16. In some embodiments, the delivery system may include a mid-hub 18. In some embodiments, the delivery system 10 may include a mid-shaft 20 extending from the proximal hub 16 to the mid-hub 18. In some embodiments, the delivery sheath 14 may extend distally from the mid-hub 18. Other configurations are also contemplated. In some embodiments, the delivery system 10 may include a side port 22. In some embodiments, the side port 22 may be in communication with the mid-shaft 20. Other configurations are also contemplated. In some embodiments, the delivery system 10 and/or the delivery lumen 12 may include a proximal segment (not shown) extending within and/or through the mid-hub 18, the mid-shaft 20, and the proximal hub 16. In some embodiments, the proximal segment may be in fluid communication with and/or may be an extension of the delivery lumen 12 of the delivery sheath 14. In some embodiments, the side port 22 may be in fluid communication with the proximal segment and/or the delivery lumen 12.

The occlusive implant system and/or the delivery system 10 may include a core member or core wire 24 slidably and/or rotatably disposed within the delivery lumen 12 (and the proximal segment, where present). The occlusive implant system may include an occlusive implant 26, which may be configured for implantation within a left atrial appendage, releasably engaged with and/or releasably attached to a distal end of the core wire 24. In at least some embodiments, the occlusive implant 26 may be a left atrial appendage closure device. In at least some embodiments, the occlusive implant 26 may be a WATCHMAN FLX™ or a WATCHMAN FLX™ Pro available from Boston Scientific Corporation. In some embodiments, a proximal end of the core wire 24 may extend proximally of a proximal end of the delivery sheath 14 and/or the proximal opening of the delivery lumen 12 for manual manipulation by a clinician or practitioner. In at least some embodiments, the delivery sheath 14 may comprise and/or may be formed from a polymeric material. In some embodiments, the delivery sheath 14 may comprise and/or may be formed from a plurality of polymeric materials. In some embodiments, the delivery sheath may comprise and/or may be formed from a combination of metallic and polymeric materials. In some embodiments, the delivery sheath 14 may include a reinforcing element, such as a mesh, a coil, a braid, etc., formed therein, embedded therein, attached thereto, etc. along at least a portion of a length of the delivery sheath 14. Other configurations are also contemplated. Some suitable, but non-limiting, examples of materials for the occlusive implant system, the core wire 24, and/or the delivery sheath 14, etc., including but not limited to metallic materials, polymeric materials, etc., are discussed below.

The occlusive implant 26 may include an expandable framework 28 (e.g., FIG. 2) configured to shift between a delivery configuration (e.g., FIG. 1), such as when the occlusive implant 26 is disposed within the delivery lumen 12 proximate the distal opening and/or within a distal portion of the delivery lumen 12, and a deployed configuration (e.g., FIG. 2) when the occlusive implant 26 is unconstrained by the delivery sheath 14.

In some embodiments, the expandable framework 28 may comprise a plurality of interconnected struts. In some embodiments, the expandable framework 28 may be compliant or semi-compliant and may generally conform to and/or be configured to sealingly engage with the shape and/or geometry of the left atrial appendage in the deployed configuration.

In some embodiments, a proximal end of the expandable framework 28 may be configured to releasably attach, join, couple, engage, or otherwise connect to the distal end of the core wire 24 (e.g., FIG. 2). In some embodiments, the proximal end of the expandable framework 28 may include a proximal hub coupled and/or non-releasably attached thereto. In some embodiments, the proximal hub may be configured to and/or adapted to releasably couple with, join to, mate with, or otherwise engage a distal end of the core wire 24. Other means of releasably coupling and/or engaging the expandable framework 28 to the distal end of the core wire 24 are also contemplated.

In some embodiments, the occlusive implant 26 may include an occlusive element 30 (e.g., a membrane, a fabric, or a tissue element, etc.) connected to, disposed on, disposed over, disposed about, or covering at least a portion the expandable framework 28. In some embodiments, the occlusive element 30 may be connected to, disposed on, disposed over, disposed about, or cover at least a portion of an outer (or outwardly facing) surface of the expandable framework 28.

In some embodiments, the occlusive element 30 may be permeable or impermeable to blood and/or other fluids, such as water. In some embodiments, the occlusive element 30 may include a polymeric membrane, a metallic or polymeric mesh, a porous or semi-porous filter-like material, or other suitable construction. In some embodiments, the occlusive element 30 prevents thrombi (e.g., blood clots, etc.) from passing through the occlusive element 30 and out of the left atrial appendage into the blood stream. In some embodiments, the occlusive element 30 promotes endothelization after implantation, thereby effectively removing the target site (e.g., the left atrial appendage, etc.) from the patient's circulatory system. Some suitable, but non-limiting, examples of materials for the occlusive element 30 are discussed below.

In some embodiments, the expandable framework 28 and/or the plurality of interconnected struts may be integrally formed and/or cut from a unitary member. In some embodiments, the expandable framework 28 and/or the plurality of interconnected struts may be integrally formed and/or cut from a unitary tubular member and subsequently formed and/or heat set to a desired shape in the deployed configuration. In some embodiments, the expandable framework 28 and/or the plurality of interconnected struts may be integrally formed and/or cut from a unitary flat member or sheet, and then rolled or formed into a tubular structure and subsequently formed and/or heat set to the desired shape in the deployed configuration. Some exemplary means and/or methods of making and/or forming the expandable framework 28 include laser cutting, machining, punching, stamping, electro discharge machining (EDM), chemical dissolution, etc. Other means and/or methods are also contemplated.

In use, the delivery sheath 14 may be advanced and/or navigated to the left atrial appendage to deliver the occlusive implant 26 thereto. In one example, the delivery sheath 14 may be advanced and/or navigated to the left atrial appendage using and/or over a guidewire. For example, the delivery sheath 14 may be advanced to the patient's left atrium and the distal end disposed adjacent to the left atrial appendage with the occlusive implant 26 disposed therein in the delivery configuration. In some embodiments, the delivery sheath 14 may include steering capability. After the distal end of the delivery sheath 14 is disposed adjacent to and/or at the left atrial appendage, the core wire 24 may be advanced distally relative to the delivery sheath 14 to advance the occlusive implant 26 out of the delivery sheath 14, where the occlusive implant 26 may shift to the deployed configuration.

While not expressly illustrated, in some embodiments, the occlusive implant system may further comprise an access device. In some embodiments, the access device may be a bi-directional steerable catheter and/or an intravascular catheter. Examples of intravascular catheters may include, but are not limited to, balloon catheters, atherectomy catheters, device delivery catheters, drug delivery catheters, diagnostic catheters, and guide catheters.

In some embodiments, the access device may be advanced and/or navigated to the left atrial appendage. In one example, the access device may be advanced and/or navigated to the left atrial appendage using and/or over a guidewire. For example, the access device may be advanced to the patient's left atrium and a distal tip disposed adjacent to the left atrial appendage. In some embodiments, the access device may include steering capability. In some embodiments, the delivery system 10 may be inserted through the access device. In some embodiments, the length of the delivery sheath 14 may be substantially equal to the length of the access device. In some embodiments, the length of the delivery sheath 14 may be slightly longer than the access device. During use, the delivery sheath 14 may be advanced within the access device with the occlusive implant 26 disposed therein in the delivery configuration. After the distal end of the delivery sheath 14 is disposed adjacent to and/or at the distal end of the access device, the core wire 24 may be advanced distally relative to the delivery sheath 14 and/or the access device to advance the occlusive implant 26 out of the delivery sheath 14 and the access device, where the occlusive implant 26 may shift to the deployed configuration.

In some embodiments, the delivery system 10, the delivery sheath 14, and/or the access device may be sized in accordance with its intended use. For example, the delivery system 10, the delivery sheath 14, and/or the access device can have a length that is in the range of about 10 to about 150 centimeters, about 25 to about 125 centimeters, about 50 to about 100 centimeters, about 25 centimeters to about 50 centimeters, about 50 to about 75 centimeters, about 75 to about 100 centimeters, etc. Other lengths are also contemplated, including but not limited to subsets of ranges disclosed herein. It is further contemplated that the outer diameter of the delivery system 10, the delivery sheath 14, and/or the access device may vary based on the use or application. In some examples, the outer diameter of the delivery system 10, the delivery sheath 14, and/or the access device may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm, about 6.5 mm, about 7 mm (or 21 French), about 8 mm, or other suitable sizes. In some embodiments, the outer diameter of the delivery system 10, the delivery sheath 14, and/or the access device may be a maximum of 5.66 mm (17 French) and is preferably smaller than 5.66 mm (17 French). Other configurations are also contemplated. In some embodiments, it is desirable for the outer diameter of the delivery system 10, the delivery sheath 14, and/or the access device to be as small as possible.

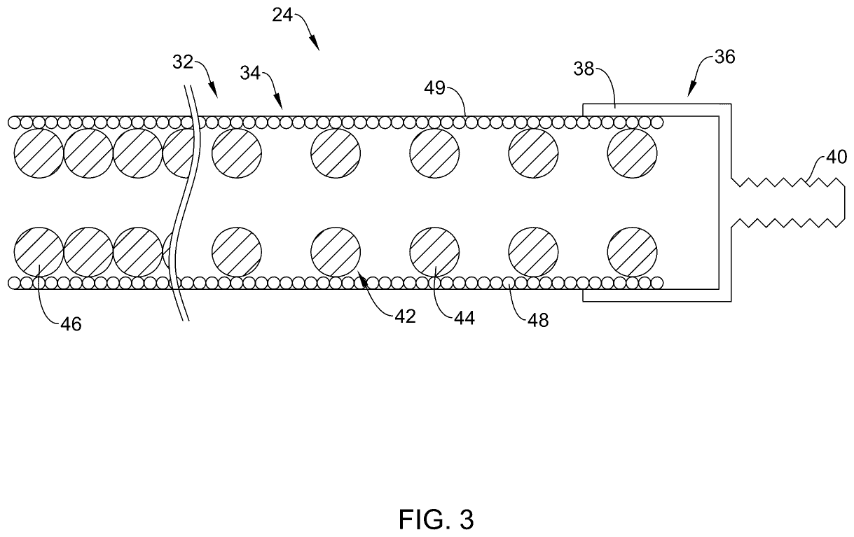

It can be appreciated that in order to efficiently secure an occlusive implant (e.g., the occlusive implant 26) to a core shaft 32 on the core wire 24 of the delivery system 10, the delivery sheath 14, and/or the access device, the core shaft 32 may include one or more structural features generally configured to engage and releasably secure the occlusive implant. For example, FIG. 3 illustrates that the core wire 24 may include a core shaft 32 having a distal end region 34. A tip member 36 may be coupled to the distal end region 34. The tip member 36 may include a collar region 38 and an occlusive implant securing region 40. The occlusive implant securing region 40 may take the form of a threaded connector configured to engage a corresponding threaded region on the occlusive implant 26.

In some instances, the core shaft 32 and tip member 36 may be manufactured to be a “one piece” construction where the tip member is machined into the end of a wire or shaft. Alternatively, the core shaft 32 may be designed such that the tip member 36 is a separate component secured to the distal end region 34 of the core shaft 32. Forming the core wire 24 in a manner where the core shaft 32 and the tip member 36 are separate components may allow for the core shaft 32 to be manufactured to have a number of desirable properties such as desirable flexibility and/or pushability (e.g., which may be more akin to the features of a catheter). For example, the core shaft 32 may include and/or be formed from a coil member 42 (e.g., coil) having a first or proximal region 46 and a second or distal region 44. Along the proximal region 46, the coil member 42 may be a closed pitch and/or tightly wound coil where individual windings of the coil member 42 contact one another. In some of these and in other instances, along the distal region 44, the coil member 42 may be an open pitch and/or spaced coil where individual winding of the coil member 42 are spaced apart. A braid 48 may be disposed over the coil member 42. In at least some instances, a sleeve or coating 49 may be disposed along the braid 48 that may cover and/or encapsulate the braid 48.

Securing the tip member 36 to the distal end region 34 of the core shaft 32 may include a suitable bonding process such as a thermal bond (e.g., reflow, overflow, etc.), adhesive bond, mechanical bond, combinations thereof, and/or the like. For example, the tip member 36 (e.g., the collar region 38 thereof) may be disposed along an outer surface of the distal end region 34 of the core shaft 32. This may include disposing the tip member 36 over the braid 48 and/or sleeve 49. The application of heat may form, for example, a thermal bond. In other words, the tip member 36 (e.g., the collar region 38 thereof) may be thermally bonded with the distal end region 34 of the core shaft 32.

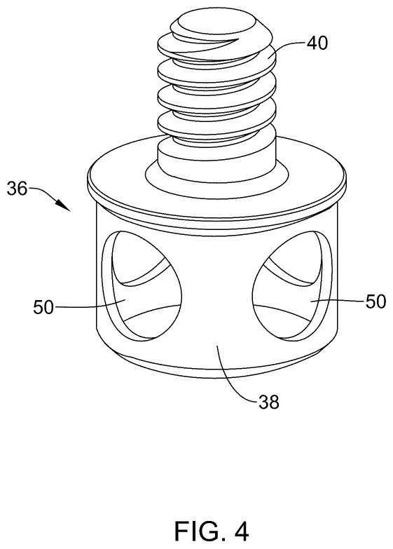

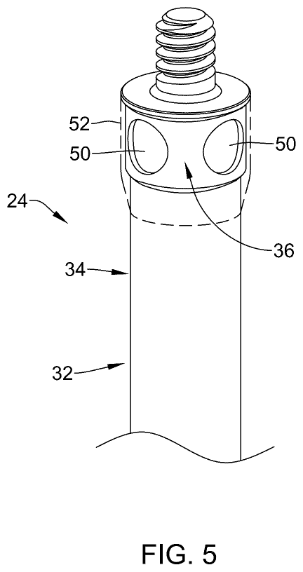

In some instances, the tip member 36 may include one or more structural features designed to improve the integrity of the bond between the tip member 36 and the core shaft 32. For example, FIG. 4 is a perspective view of the tip member 36. Here it can be seen that the collar region 38 may have one or more apertures 50 formed therein. The apertures 50 may have a variety of forms, configurations, and/or arrangements. Some of the contemplated variations include ratios of aperture surface area to non-aperture surface areas in the range of 1:8 to 8:1 (e.g., 1:1, 1:2, 1:4, 1:8, 2:1, 4:1, 8:1, etc.). For example, the apertures 50 may be round, oval, have a geometric shape, etc. The apertures 50 may allow for material of the core shaft 32 such as the sleeve 49 to flow into during the formation of a thermal bond. In other words, a portion of the core shaft 32 (e.g., such as the sleeve 49) may extend at least partially into at least some of the apertures 50. This, for example in addition to the thermal bond, may help to form a mechanical bond between the tip member 36 and the core shaft 32. In some instances, an outer sleeve 52 may be disposed over the collar region 38 as shown in FIG. 5. This may include disposing the outer sleeve 52 along a portion of the distal end region 34 of the core shaft 32. In at least some instances, the outer sleeve 52 may also flow into the apertures 50, for example during a thermal bonding process, and help to mechanically bond the tip member 36 to the core shaft 32. The outer sleeve 52 may include suitable materials such as those disclosed herein. For example, the outer sleeve 52 may be formed from a polyether block amide (e.g., PEBAX®), a polyamide, a nylon, a nylon-12 (e.g., VESTAMID®), a high-density polyethylene, a low-density polyethylene, a urethane, combinations thereof, and/or the like.

While the forgoing disclosure is directed at core wires 24 for delivering occlusive implants where a tip member 36 is secured thereto using a thermal and/or mechanical bond, it can be appreciated that similar tipping/bonding strategies can be applied to a number of different devices. In other words, this disclosure is not intended to be limited to just core wires for delivering occlusive implants as other devices are contemplated. For example, catheters, balloon catheters, delivery systems, and/or the like may include structural features such as tips or nose cones. In at least some instances, such structures may be manufactured to include features similar to the apertures 50 so that these structures can be secured to a distal end region of a shaft in a manner analogous to what is disclosed herein.

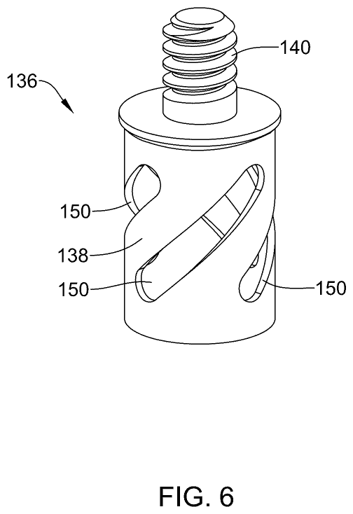

FIG. 6 illustrates another example tip member 136 that may be similar in form and function to other tip members disclosed herein. The tip member 136 may include a collar region 138 and an occlusive implant securing region 140. A plurality of slots or apertures 150 may be disposed along the collar region 138. In this example, the slots 150 may be disposed at an angle (e.g., angled relative to the longitudinal axis of the collar region 138). This may increase the torque-transmitting capability (e.g., in the direction of the slots 150) of the tip member 136 and/or impact (e.g. include) the flexibility of the tip member 136. A variety of angles, arrangements, and/or configurations are contemplated for the slots 150. The tip member 136 may be secured to a core shaft (e.g., the core shaft 32) in a similar manner to what is disclosed herein. For example, the tip member 136 may be disposed over the braid 48 and/or sleeve 49 of the core shaft 32. The application of heat may form, for example, a thermal bond. This may include allowing the material of the core shaft 32 such as the sleeve 49 to flow into the slots 150 during the formation of a thermal bond, which may also help to form a mechanical bond between the tip member 136 and the core shaft 32. In at least some instances, an outer sleeve (e.g., the outer sleeve 52) may also be disposed over the collar region 138 in a manner similar to what is disclosed herein.

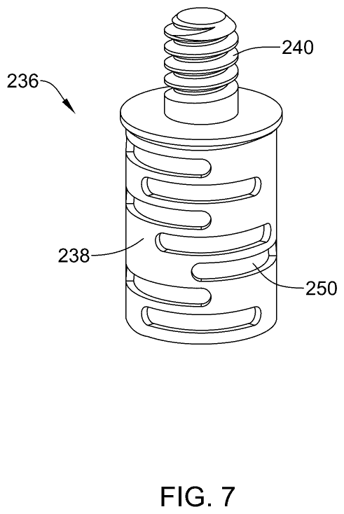

FIG. 7 illustrates another example tip member 236 that may be similar in form and function to other tip members disclosed herein. The tip member 236 may include a collar region 238 and an occlusive implant securing region 240. A plurality of slots or apertures 250 may be disposed along the collar region 238. In this example, the slots 250 may be disposed at an angle that is substantially normal to the longitudinal axis of the collar region 238. The slots 250 may change the bending stiffness along the collar region 238 (e.g., increasing flexibility) while allowing for efficient torque transition along the tip member 236. A variety of arrangements are contemplated for the slots 250 such as groups of two or more slots, differing slot thicknesses, etc. For example, the slots 250 may have a slot width on the order of about 5-45% of the diameter of the collar region 238. The slot width, the number of slots 250 per unit length, the slot height, etc. can vary along the length of the collar region 238. The tip member 236 may be secured to the core shaft 32 in a similar manner to what is disclosed herein. For example, the tip member 236 may be disposed over the braid 48 and/or sleeve 49 of the core shaft 32. The application of heat may form, for example, a thermal bond. This may include allowing the material of the core shaft 32 such as the sleeve 49 to flow into the slots 250 during the formation of a thermal bond, which may also help to form a mechanical bond between the tip member 236 and the core shaft 32. In at least some instances, an outer sleeve (e.g., the outer sleeve 52) may also be disposed over the collar region 238 in a manner similar to what is disclosed herein.

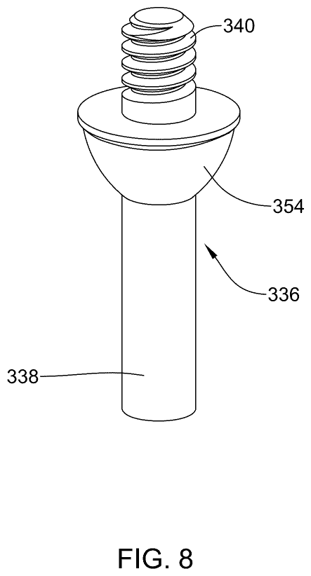

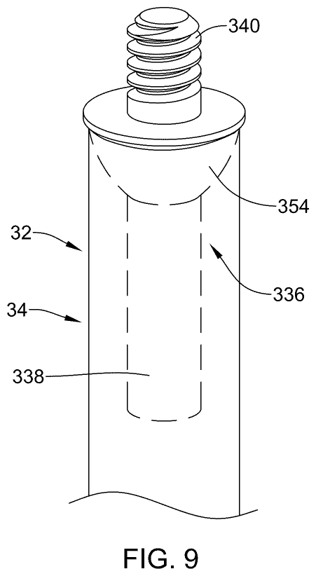

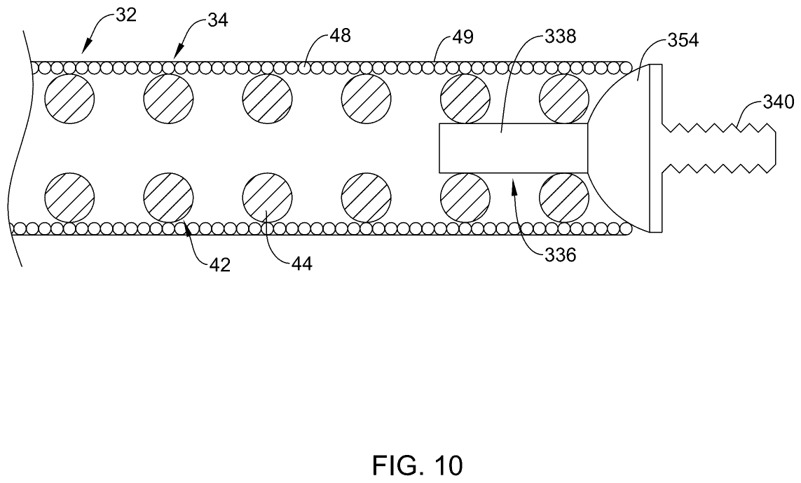

FIG. 8 illustrates another example tip member 336 that may be similar in form and function to other tip members disclosed herein. The tip member 336 may include a collar or stem region 338, an occlusive implant securing region 340, and a transition region 354. In some instances, the stem region 338 may be configured to be inserted into the distal end region 34 of the core shaft 32 as shown in FIGS. 9-10. When doing so, the stem region 338 may be disposed within the core shaft 32 and/or otherwise disposed along an inner surface of the distal end region 34 of the core shaft 32. In some instances, the stem region 338 may be secured to the core shaft 32 (e.g., at the coil member 42, braid 48, and/or sleeve 49), for example using a thermal bond, adhesive bond, combinations thereof, and/or the like. In some of these and in other instances, the transition region 354 may be be secured to the core shaft 32 (e.g., at the coil member 42, braid 48, and/or sleeve 49) for example using a thermal bond, adhesive bond, combinations thereof, and/or the like.

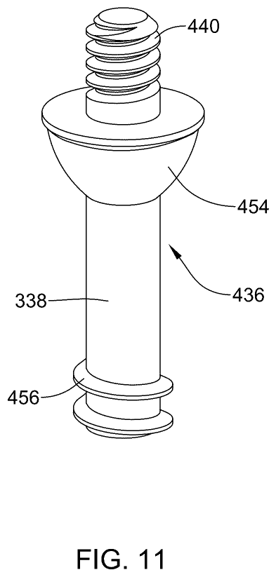

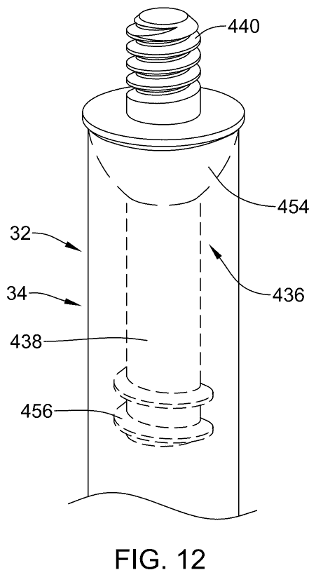

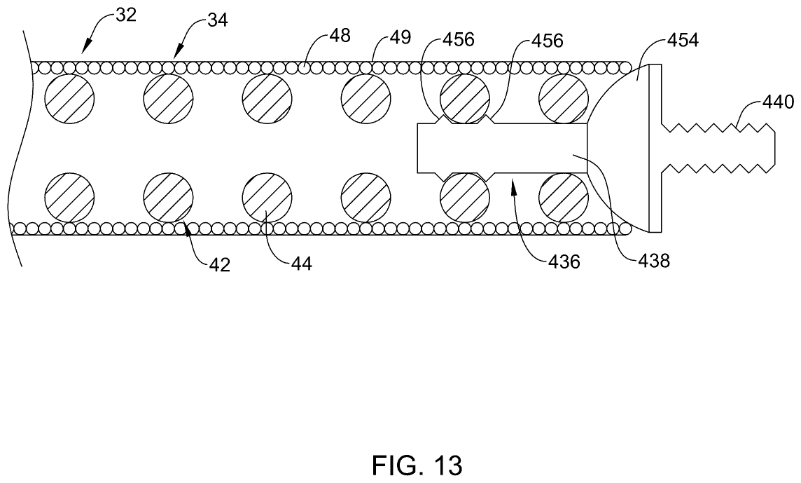

FIG. 11 illustrates another example tip member 436 that may be similar in form and function to other tip members disclosed herein. The tip member 436 may include a collar or stem region 438, an occlusive implant securing region 440, and a transition region 454. One or more threads 456 may be disposed along the stem region 438. The tip member 436 may be configured to be secured to the distal end region 34 of the core shaft 32 as shown in FIGS. 12-13. When doing so, the stem region 438 may be disposed within the core shaft 32 and/or otherwise disposed along an inner surface of the distal end region 34 of the core shaft 32. This may include threadably engaging the threads 456 with the open pitch region of the coil member 42. This may form a mechanical bond between the tip member 436 (e.g., the stem region 438) and the core shaft 32. In some of these and in other instances, the stem region 438 may also be secured to the core shaft 32 (e.g., at the coil member 42, braid 48, and/or sleeve 49), for example using a thermal bond, adhesive bond, combinations thereof, and/or the like. In some of these and in other instances, the transition region 454 may be secured to the core shaft 32 (e.g., at the coil member 42, braid 48, and/or sleeve 49) for example using a thermal bond, adhesive bond, combinations thereof, and/or the like.

The materials that can be used for the various components of the delivery system 10 (and/or other delivery systems disclosed herein) may include those commonly associated with medical devices. For simplicity purposes, the following discussion makes reference to the delivery system 10. However, this is not intended to limit the devices and methods described herein, as the discussion may be applied to other delivery systems disclosed herein.

The delivery system 10 and/or other components of the delivery system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as VESTAMID®, GRILAMID® available from EMS American Grilon, and/or the like), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the delivery system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the delivery system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the delivery system 10 to achieve the same result.

In some embodiments, a degree of Magnetic Resonance Imaging (MRI) compatibility is imparted into the delivery system 10. For example, the delivery system 10, or portions thereof, may be made of a material that does not substantially distort the image and create substantial artifacts (e.g., gaps in the image). Certain ferromagnetic materials, for example, may not be suitable because they may create artifacts in an MRI image. The delivery system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device for use in delivering an occlusive implant, the medical device comprising:
a core shaft having a distal end region;
a tip member coupled to the distal end region, the tip member having a collar region and an occlusive implant securing region;
wherein the collar region includes a plurality of apertures formed therein;
wherein the collar region is bonded to the distal end region such that a portion of the distal end region of the core shaft forms a mechanical bond with the collar region; and
a sleeve disposed over the collar region.

2. The medical device of claim 1, wherein the core shaft includes a coil.

3. The medical device of claim 2, further comprising a braid disposed over the coil.

4. The medical device of claim 2, wherein the coil includes an open pitch region along at least a section of the distal end region.

5. The medical device of claim 1, wherein the collar region extends along an outer surface of the distal end region of the core shaft.

6. The medical device of claim 5, wherein the collar region is thermally bonded with the distal end region of the core shaft.

7. The medical device of claim 6, wherein the mechanical bond with the collar region is formed by the core shaft extending at least partially into at least some of the apertures of the collar region.

8. The medical device of claim 1, wherein the collar region extends along an inner surface of the distal end region of the core shaft.

9. The medical device of claim 8, wherein the collar region is threadably engaged with the inner surface of the distal end region of the core shaft.

10. The medical device of claim 1, wherein the apertures include one or more round or oval openings.

11. The medical device of claim 1, wherein the apertures include one or more slots oriented substantially normal relative to a longitudinal axis of the collar region.

12. The medical device of claim 16, wherein the apertures include one or more slots angled relative to a longitudinal axis of the collar region.

13. A medical device for use in delivering an occlusive implant, the medical device comprising:
a core shaft having a distal end region;
wherein the core shaft includes a coil;
wherein the coil includes an open pitch section adjacent to the distal end region of the core shaft;
a tip member coupled to the distal end region, the tip member having a collar region configured to attach the tip member to the core shaft and an occlusive implant securing region configured to be attached to an occlusive implant;
wherein the collar region is bonded to the distal end region such that a portion of the distal end region of the core shaft forms a mechanical bond with the collar region; and
a sleeve disposed over the collar region.

14. The medical device of claim 13, further comprising a braid disposed over the coil.

15. The medical device of claim 13, wherein the collar region extends along an outer surface of the distal end region of the core shaft.

16. The medical device of claim 15, wherein the collar region is thermally bonded with the distal end region of the core shaft.

17. The medical device of claim 13, wherein the collar region extends along an inner surface of the distal end region of the core shaft.

18. The medical device of claim 17, wherein the collar region is threadably engaged with the open pitch section of the coil.

19. The medical device of claim 13, wherein the collar region includes a plurality of apertures formed therein.

20. A method for manufacturing a medical device, the method comprising:
engaging a collar region of a tip member with a distal end region of a core shaft;
wherein the collar region includes a plurality of apertures formed therein; and
bonding the collar region with the distal end region such that a portion of the distal end region of the core shaft extends into at least some of the apertures and forms a mechanical bond with the collar region.
